(19) United States
(12) Patent Application Publication
(10) Pub. No.: US 2011/0009726 A1
(43) Pub. Date: Jan. 13, 2011

(54) TAPE CASSETTE FOR A MEDICAL HANDHELD DEVICE AND BLOOD SUGAR MEASURING SYSTEM

(76) Inventor: Herbert Harttig, Neustadt (DE)

Correspondence Address: BOSE MCKINNEY & EVANS LLP, 111 Monument Circle, Suite 2700, INDIANAPOLIS, IN, 46204, US

(21) Appl. No.: 12/833,961

(22) Filed: Jul. 10, 2010

Related U.S. Application Data
(63) Continuation of application No. PCT/EP2008/010982, filed on Dec. 20, 2008.

(30) Foreign Application Priority Data
Jan. 11, 2008 (EP) 08 000 435.1

(51) Int. Cl. A61B 5/00; B23P 11/00; B29C 45/16

(52) U.S. Cl. 600/365; 29/428; 29/460; 264/241

(57) ABSTRACT

A tape cassette for a medical handheld device. The inventive tape cassette includes a carrier tape with a plurality of functional elements which may take the form of test fields for analyzing a sample of a human or animal body fluid and/or a plurality of lancets. During use, the carrier tape slides past friction points. To reduce friction between the carrier tape and at least one of the friction points, the at least one friction point includes a surface made of a fluoropolymer. The tape cassette may have a housing that includes a sealed compartment with a tape exit opening wherein the friction point is disposed proximate the tape exit opening. A cover element may also be positioned to cover the tape exit opening. The cover element may also include a fluoropolymer material engageable with the carrier tape. A method of manufacturing such tape cassettes is also disclosed.

### RELATED APPLICATIONS

This is a continuation application of International Patent Application Serial Number PCT/EP2008/010982 filed Dec. 20, 2008 which claims priority to EP 08000435.1 filed Jan. 11, 2008, both of which are hereby incorporated by reference in their entirety.

### BACKGROUND OF THE INVENTION

The invention relates to a tape cassette for a handheld medical device that contains a carrier tape for analyzing a sample of human or animal body fluid.

A tape cassette for use with a handheld medical device is known from EP 1 424 040 A1. It is known to provide such tape cassettes with a carrier tape having functional elements that take the form of test fields for analyzing a sample of body fluid. The test fields are inserted into a measuring device that is used to determine the presence or the concentration of an analyte such as glucose. Tape cassettes of this type are useful to diabetics who have to carry out multiple daily measurements of the glucose concentration of a sample of body fluid, usually blood or interstitial fluid, obtained by a lancet puncture.

It is also known to provide tape cassettes with a carrier tape that carries both test fields and lancets as functional elements. Tape cassettes of this type are intended for integrated handheld devices that can be used to carry out both a measurement of a sample of body fluid and a lancet puncture for obtaining the sample. Simpler blood sugar measuring systems, however, often utilize two separate handheld devices, namely, a puncturing device and a measuring device. For puncturing devices of this type, it is known to provide tape cassettes whose carrier tapes carry lancets as functional elements.

For medical handheld devices such as those used by diabetics, it is important that the transport of the tape be as easy as possible to provide a user-friendly device. In simpler handheld devices, the transport of the tape is performed manually by the user. More complex handheld devices have an electric motor to successively move the functional elements of the carrier tape into a usage position.

### SUMMARY OF THE INVENTION

The present invention provides a means to simplify the transport of a carrier tape of a medical handheld device.

In accordance with exemplary embodiments, the carrier tape of a tape cassette slides past at least one friction point having a fluoropolymer in order to reduce friction during movement of the carrier tape. Surprisingly, this simple measure allows the tape friction to be reduced significantly.

Instead of seeking reduction of the frictional force by changing the guidance of the tape with a reduced wrap-around angle, or reducing the force pressing the carrier tape against the friction points, or optimizing the material of the carrier tape with regard to its frictional properties, these teachings take a very different approach.

The surfaces of the friction points of the tape cassette or medical handheld device in which the tape cassette is received can be formed with a material containing fluoropolymers to thereby advantageously reduce the friction. The fluoropolymer can be applied in the form of a coating such as a varnish or film. It is also feasible to manufacture the entire component from a fluoropolymer or to use a two-component injection molding procedure employing a fluoropolymer material in combination with another plastic material.

Embodiments disclosed herein advantageously provide a substantial reduction of tape friction by a relatively simple means without changing the guidance of the tape or the mechanical transport mechanism. In manually-operated handheld devices, user convenience is improved by significantly lowering the force needed to transport the tape. In devices with an electric motor, a lower power motor and consequently a smaller and less expensive motor can be used. Moreover, the energy consumption of the motor is reduced thereby allowing the use of smaller, lighter-weight, and less expensive batteries.

As used herein, fluoropolymers are understood to include all organic and silicon-organic polymers whose monomers contain covalently bound fluorine and which are provided pure or as a mixture or alloy or copolymer including other polymers that do not contain fluorine.

The fluoropolymer used in accordance with these teachings can contain, for example, polytetrafluoroethylene (PTFE) or polyvinylidene fluoride (PVDF), preferably at least 10% by weight, for example 10% by weight to 40% by weight. A mixture of polypropylene and PFTE is a particular preferred fluoropolymer.

Test fields for analyzing a sample of a human or animal body fluid usually contain sensitive detection reagents. Such detection reagents may, for example, be used for photometric determination of an analyte concentration. A tape cassette with a carrier tape that carries test fields for analyzing a sample of body fluid as functional elements therefore usually needs to have a sealed housing to prevent the unused test fields from being adversely affected by moisture. A tape exit opening having a seal is often used with such tape cassettes and is a substantial source of friction. These teachings can therefore be used in particularly advantageous fashion with a friction point that is situated at the tape exit opening of a cassette housing.

In some embodiments, the tape exit opening can be sealed with a sealing lip, in particular, with an elastomeric sealing lip having a surface made of fluoropolymer. In other embodiments, the surface made of fluoropolymer can be applied as varnish. It is also feasible to use a fluoropolymer-containing elastomer as the material for the sealing lip.

Further details and advantages of the invention are illustrated in the drawings and described below. The different features may be advantageously employed either alone or in various combinations.

### BRIEF DESCRIPTION OF THE DRAWINGS

The above-mentioned aspects and the manner of obtaining them will become more apparent and the invention itself will be better understood by reference to the following description of embodiments of the invention taken in conjunction with the accompanying drawings, wherein:

FIG. 1 is a perspective view of an exemplary embodiment of a handheld device for measuring a glucose concentration;

FIG. 2 is a schematic perspective view showing a tape cassette for the handheld device shown in FIG. 1;

FIG. 3 is an enlarged schematic cross sectional view of an exemplary embodiment of a tape exit opening of a tape cassette;

FIG. 4 is an enlarged schematic cross sectional view of another exemplary embodiment of a tape exit opening; and

FIG. 5 is a schematic diagram of a mechanism for reducing the tape friction at the tape exit opening of a tape cassette.

Corresponding reference characters indicate corresponding parts throughout the several figures.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of the present invention.

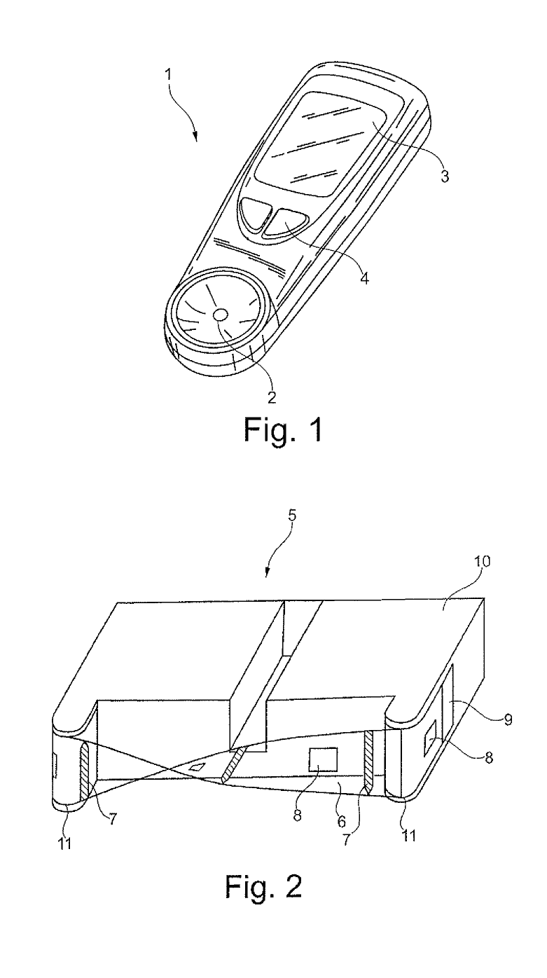

FIG. 1 shows an exemplary embodiment of a medical handheld device 1 for determining the concentration of an analyte in a sample of human body fluid. Handheld devices 1 of this type are used, in particular, by diabetics in order to determine the concentration of glucose in a sample of blood or interstitial fluid.

The handheld device 1 has a device opening 2 against which a body part is pressed in order to draw a sample of body fluid. A measurement result is displayed with a display facility 3, for example, a liquid crystal display. Operating elements 4 in the form of buttons are provided for the operation of measuring device 1.

The measuring device 1 shown in FIG. 1 has a compartment on its backside, into which a tape cassette 5 shown in FIG. 2 can be inserted. The measuring device 1 and the tape cassette 5 jointly form a blood sugar measuring system. The tape cassette 5 contains a carrier tape 6 that carries, as functional elements, both lancets 7 and test fields 8. Lancets 7 are used to obtain a sample of body fluid by means of a puncture and test fields 8 are used to analyze a sample of the body fluid. The measuring device 1 contains a tape conveying facility (not shown), in order to consecutively position the functional elements 7, 8 in a usage position. Lancets 7 can thereby be used to puncture a body part that is positioned against the housing opening 2 and a test field 8 can be used to take up and analyze by photometric means a sample of body fluid emanating from the puncture wound thus generated. The test fields 8 contain detection reagents that effect a concentration-dependent color change of the test field 8 upon contact with a sample. This color change can be measured and analyzed with a measuring unit that is contained in the measuring device 1.

During tape transport, the carrier tape 6, which can, for example, be made of polyester, slides past various friction points located on the tape cassette 5. Such friction points may also be located on the measuring device 1. In the exemplary embodiment shown in FIG. 2, friction points are present at a tape exit and a tape entry opening 9 of the cassette housing 10 as well as on tape guiding elements 11, along which the carrier tape 6 slides. In order to reduce friction, the friction points of the exemplary embodiment shown have a surface made of a fluoropolymer. The fluoropolymer can, for example, be a coating, in particular a varnish or a film/foil. It is also feasible for the fluoropolymer to have been applied in a two-component injection molding process during the manufacture of the cassette housing.

In the illustrated embodiment, the carrier tape 6 is twisted by 180° between its exit from the cassette housing 10 and its re-entry. By this means, the orientation of the lancets 7 can be aligned with respect to the device opening 2 for a puncture. However, it is also feasible to guide the carrier tape 6 without twisting.

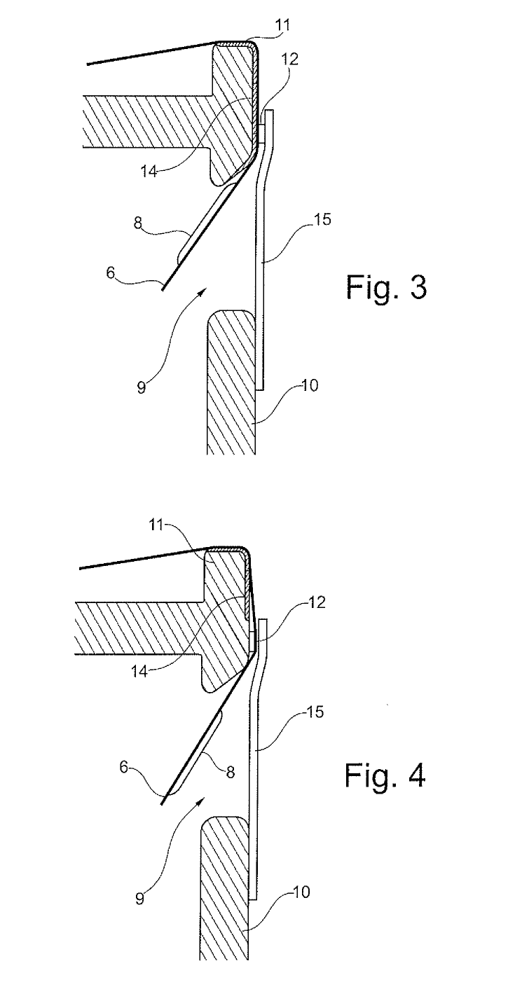

A significant part of the tape friction originates in the area of the openings 9 of the cassette housing 10 through which the carrier tape 6 is guided. Since the test fields 8 are moisture-sensitive, the carrier tape 6 or at least the tape section with unused functional elements 8 is arranged in a sealed housing or a sealed part of the cassette housing 10, and, for this purpose, the tape exit opening 9 is provided with a sealing element 12 like the one shown in FIG. 3. Sealing element 12 is preferably designed as a sealing lip made of an elastomeric material. Sealing elements of this type can lead to very high friction, especially when the functional elements 7, 8 are guided past them. In order to reduce friction, the sealing element 12 in the illustrated embodiment has a surface made of a fluoropolymer. The fluoropolymer can be applied as a coating such as a varnish. It is also feasible for the sealing element 12 to be made of an elastomeric fluoropolymer.

FIG. 3 shows a detailed view of the tape exit opening 9 of the tape cassette 1. In order to reduce friction, the test fields 8 are arranged on the side of the carrier tape 6 facing away from the sealing element 12. By this means, the relatively rough test fields 8 can slide along a friction point 14 that is formed by the cassette housing 10 and has a surface made of fluoropolymer. The relatively smooth backside of the tape slides along the friction point formed by the sealing element 12. The sealing element 12 is attached to a cover element 15 that covers the tape exit opening 9. The cover element 15 may be a foil. The cover element 15, which may be made of aluminum, plastic or a composite, is exposed to the force of a spring element 19 that is not shown, and presses the sealing element 12 that is arranged at the end thereof against the carrier tape 6 in a spring-like fashion.

In one embodiment of the present invention, the friction point 14 of the cassette housing 10 can be provided with a fluoropolymer surface in a two-component injection molding process. The two-component injection molding process can be carried out, for example, by inserting into an injection mold for a cassette housing 10 a removable core that forms cavities for forming the friction points. The cavities are filled during injection molding, whereby a mixture of polypropylene and 20% by weight PTFE micro-powder can be used, which can be procured, for example, from Dyneon GmbH & Co.KG, Neuss, Germany. For a second injection shot, the removable core is exchanged and the injection molding of the housing is completed with polypropylene containing no PTFE fraction.

In other exemplary embodiments, the friction point 14 can be formed by injection molding behind a PTFE foil.

If the fluoropolymer surface is applied as varnish, it is advantageous to pre-treat the cassette housing 10 to attain better adhesion of the varnish. For example, a polypropylene housing can be treated by a plasma treatment, in particular, atmospheric plasma or any other suitable activation method, in order to attain better adhesion of the varnish. PTFE-containing varnishes are preferred for the friction points of the housing and the sealing element 12. Suitable varnishes are commercially available, for example, from Fuchs Lubritech, Weilerbach, Germany, under the names, gleitmo SFL9062K and gleitmo SFL9460K1.

Another embodiment of a tape entry or tape exit opening 9 of a tape cassette 5 is shown in FIG. 4. The exemplary embodiment of FIG. 4 differs from the exemplary embodiment shown in FIG. 3 in that the sealing element is not attached to the cover element 15, but rather to the cassette housing 10. For this reason, the functional elements 7, 8 are arranged on the other side of the carrier tape 6 whereby they face the cover element 15. Since the cover element 15 touches against the carrier tape 6 and thereby forms a friction point in this exemplary embodiment, the cover element 15 also has a surface made of a fluoropolymer. The cover element 15 can, for example, be an aluminum foil to which a fluoropolymer is applied as a varnish. For example, a PTFE-containing varnish can be used. In order to improve the adhesion of the varnish, the cover element 15 can be subjected to an activation process, e.g., a plasma treatment, prior to application of the varnish.

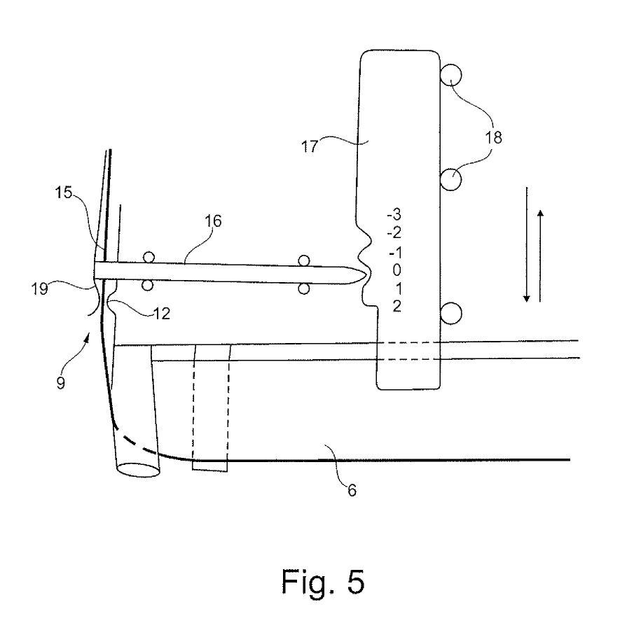

FIG. 5 shows a schematic diagram of a mechanism that can be used to attain additional reduction of tape friction at the tape exit opening 9 of a tape cassette 5. The mechanism shown in FIG. 5 generates a force which presses the carrier tape 6 against the sealing element 12 of the tape exit opening 9 by the cover element 15 to be reduced intermittently during tape transport by an actuation element 16. Actuation element 16 is coupled to the tape transport mechanism and intermittently reduces the load on the cover element 15 covering the tape exit opening 9 by lifting a spring element 19 that acts on the cover element 15. The actuation element 16 can, for example, be a slider or a plunger that is moved by an actuator that is activated according to need. Preferably, the actuation element 16 is driven by the tape transport mechanism of the device, for example, by means of a link motion. FIG. 5 schematically shows a driving element 17 of the measuring device 1 that is mobile along the direction of the arrows shown in FIG. 5 and is guided in the process by guiding elements 18. In a motion of this type, the actuation element 16 follows a control curve on the surface of the driving element 17 such that the spring element 19 is lifted intermittently and the tape friction is thereby reduced.

The actuation element 16 can be coupled to a tape conveying facility that moves the carrier tape 6 in its longitudinal direction by reeling it. The actuation element can alternatively be coupled to a transport mechanism that is used to move the carrier tape 6 transverse to its longitudinal direction. Such transverse movement can, for example, be employed with the device housing 2 shown in FIG. 1 in order to take up a sample or generate a puncture wound.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE NUMBERS

1 Handheld device/measuring device
2 Device opening
3 Display facility
4 Buttons
5 Tape cassette
6 Carrier tape
7 Lancet
8 Test field
9 Tape exit opening
10 Cassette housing
11 Tape guiding element
12 Sealing element
14 Friction point
15 Cover element
16 Actuation element
17 Driving element
18 Guiding element
19 Spring element

What is claimed is:

1. A tape cassette for a medical handheld device, the tape cassette comprising:
a carrier tape having disposed thereon a plurality of functional elements, wherein the functional elements include at least one of either a plurality of test fields for analyzing a sample of a human or animal body fluid or a plurality of lancets, whereby, during use, the carrier tape slidingly engages friction points disposed on the tape cassette, and wherein
at least one of the friction points includes a surface made of a fluoropolymer to thereby reduce friction between the carrier tape and the at least one friction point.

2. The tape cassette according to claim 1 wherein the fluoropolymer contains a material selected from the group consisting of PTFE and PVDF.

3. The tape cassette according to claim 1 wherein the fluoropolymer is a coating.

4. The tape cassette according to claim 3 wherein the fluoropolymer is applied as varnish.

5. The tape cassette according to claim 3 wherein the fluoropolymer is applied as a foil.

6. The tape cassette according to claim 1 wherein the at least one friction point having a surface made of a fluoropolymer is formed in a two-component injection molding process.

7. The tape cassette according to claim 1 wherein the tape cassette includes cassette housing defining a tape exit opening and wherein the at least one friction point having a surface made of a fluoropolymer is positioned proximate the tape exit opening.

8. The tape cassette according to claim 7 wherein the at least one friction point having a surface made of a fluoropolymer is formed by a sealing lip positioned proximate the tape exit opening.

9. The tape cassette according to claim 7 wherein the at least one friction point having a surface made of a fluoropolymer is formed by a cover element that covers the tape exit opening.

10. A blood sugar measuring system comprising:
a handheld device containing a carrier tape;
the carrier tape having disposed thereon a plurality of functional elements wherein the plurality of functional elements include at least one of either a plurality of test fields for analyzing a sample of a body fluid or a plurality of lancets, and
a transport mechanism that is operable to move the plurality of functional elements successively into a usage position, wherein the carrier tape slidingly engages friction points when moved by the transport mechanism; and
wherein at least one of the friction points includes a surface made of a fluoropolymer to thereby reduce friction between the carrier tape and the at least one friction point.

11. The tape cassette according to claim 10 wherein the fluoropolymer contains a material selected from the group consisting of PTFE and PVDF.

12. The tape cassette according to claim 10 wherein the at least one friction point having a surface made of a fluoropolymer is formed in a two-component injection molding process.

13. The tape cassette according to claim 12 wherein the at least one friction point having a surface made of a fluoropolymer is formed by a sealing lip positioned proximate a tape exit opening.

14. The tape cassette according to claim 12 wherein the at least one friction point having a surface made of a fluoropolymer is formed by a cover element that covers a tape exit opening.

15. A method of manufacturing a tape cassette for use with a medical handheld device comprising:
forming a cassette housing;
supporting a carrier tape including a plurality of functional elements on the cassette housing, the carrier tape being moveable relative to the cassette housing wherein the cassette housing defines at least one friction point having a surface slidingly engageable with the carrier tape during relative movement between the carrier tape and cassette housing; and
forming a surface to define the at least one friction point with a material including a fluoropolymer to thereby reduce friction between the surface and the carrier tape.

16. The method of claim 15 wherein said plurality of functional elements comprises a plurality of test fields for analyzing a sample of a human or animal body fluid.

17. The method of claim 15 wherein said plurality of functional elements comprises a plurality of lancets.

18. The method of claim 15 wherein the material including a flouropolymer includes PTFE.

19. The method of claim 15 wherein the material including a flouropolymer includes PVDF.

20. The method of claim 15 wherein the step of forming a surface to define the at least one friction point includes coating the cassette housing with a fluorpolymer.

21. The method of claim 15 wherein the step of forming a surface to define the at least one friction point includes injection molding the cassette housing with a fluoropolymer foil forming the surface defining the at least one friction point.

22. The method of claim 15 wherein the cassette housing is formed in a multi-component injection molding process that includes forming the at least one friction point by injection molding a first material containing a fluoropolymer and forming at least a portion of the cassette housing by injection molding a second material without a fluoropolymer.

23. The method of claim 15 wherein the cassette housing includes a sealed part for storing an unused portion of the carrier tape, the cassette housing further defining a tape exit opening through which the carrier tape exits the sealed part, the at least one friction point being disposed proximate the tape exit opening and wherein the method further comprises:
positioning a cover element to cover the tape exit opening and disposing an elastomeric fluoropolymer sealing element engageable with the carrier tape on the cover element.

24. The method of claim 15 wherein the cassette housing includes a sealed part for storing an unused portion of the carrier tape, the cassette housing further defining a tape exit opening through which the carrier tape exits the sealed part, the at least one friction point being disposed proximate the tape exit opening and wherein the method further comprises:
positioning an aluminum foil to cover the tape exit opening and applying a varnish including a fluoropolymer to the aluminum foil wherein the varnish is engageable with the carrier tape.
